(19) United States
(12) Patent Application Publication
(10) Pub. No.: US 2024/0417572 A1
(43) Pub. Date: Dec. 19, 2024

(54) EMBOSSING VARNISHES WITH ALIPHATIC PHOTOINITIATORS AND BIO-BASED MICROSTRUCTURE SYSTEMS

(71) Applicant: Joanneum Research Forschungsgesellschaft mbH, Graz (AT)

(72) Inventors: Dieter Nees, Thannhausen (AT); Stephan Ruttloff, Weiz (AT); Johannes Götz, Gleisdorf (AT); Anja Haase, Graz (AT); Ursula Palfinger, Gleisdorf (AT); Barbara Stadlober, Graz (AT)

(73) Assignee: Joanneum Research Forschungsgesellschaft mbH, Graz (AT)

(21) Appl. No.: 18/704,619

(22) PCT Filed: Oct. 26, 2021
(86) PCT No.: PCT/EP2021/079640

§ 371 Date: Apr. 25, 2024

(51) Int. Cl. C09D 5/16; C09D 135/02; C12M 1/00; C12M 3/06

(52) U.S. Cl. CPC C09D 5/1668; C09D 135/02; C12M 23/16; C12M 23/20

(57) ABSTRACT

An embossing varnish for micro- or nanostructured surface layers, in particular a UV-NIL embossing varnish containing a UV-curable compound and an aliphatic photoinitiator, an article with a micro- or nanostructured surface layer on a support, wherein the surface layer is the embossed and UV-cured embossing varnish; a method for producing the article with a micro- or nanostructured surface layer, in particular from bio-based compounds; and the use of the article with a micro- or nanostructured surface layer as a microfluidic structure or film with anti-reflection, flow friction reduction or adhesion effect.

### FIELD OF THE INVENTION

The present invention relates to an embossing varnish for micro- or nanostructured surface layers, in particular a UV-NIL embossing varnish containing a UV-curable compound and an aliphatic photoinitiator; an article with a micro- or nanostructured surface layer on a support, wherein the surface layer is the embossed and UV-cured embossing varnish; a method for producing the article with a micro- or nanostructured surface layer; and the use of the article.

### STATE OF THE ART

In many microfluidic-based applications, the biocompatibility or compatibility of the micro- and nanostructured substrates is a decisive factor. For example, when cultivating living cells in microfluidic applications, such as in lab-on-chip or organ-on-chip systems, the viability of these cells on the imprinted substrates is essential. The microfluidic systems are usually produced by embossing the nano- and microstructures using ultraviolet nanoimprint lithography (UV-NIL). Photoinitiators that absorb UV light and generate radicals to start the free radical polymerisation reaction are used to harden the embossing lacquers. Petrochemical aromatic photoinitiators are used in all commercially used UV-NIL embossing varnishes. While the UV-curable oligomers and monomers used to produce the structured substrate polymers often have good cell compatibility, these photoinitiators, which are only used in low concentrations, are usually cytotoxic and impair cell growth or lead to cell death.

Fluorescence is often used as a measurement method in microfluidics-based applications to detect selective reactions in the course of detecting certain biomolecules. The aromatic rings contained in all common petrochemical photoinitiators generally have a high potential for fluorescence. Even after UV curing, these aromatic rings remain in the UV-imprinted coatings in the form of photoreaction products or fragments thereof and lead to interference signals in fluorescence-based analysis methods due to their autofluorescence, which reduces the detection sensitivity.

The radical polymerisation of vinyl C═C double bonds, for example in (meth)acrylates, using α-ketocarboxylic acids or their esters, pyruvic acid ethyl esters and the aliphatic α-diketones 2,3-butanedione and 2,3-pentanedione as photoinitiators is known in the prior art (WO 2019/020805 A1; E. A. Lissi and M. V. Encina in Polymerisation Photosensitized by Carbonyl Compounds, Journal of Polymer Science: Polymer Chemistry Edition, Vol. 17, 2791-2803 (1979). This prior art merely discloses the production of unstructured polymers or smooth polymer coatings, but does not relate to the technical field of embossing coatings and therefore gives no indication of the usability of the polymerisation systems or photoinitiators mentioned therein in UV-NIL embossing methods.

### Problems to be Solved by the Invention

Due to the disadvantages of the state of the art, there is a need for improved UV-NIL embossing coatings.

It is therefore the object of the present invention to provide embossing lacquers with photoinitiators which are non-toxic and thus compatible with the skin or do not impair cell growth in microfluidic systems.

Other objects include the provision of embossing varnishes with photoinitiators that do not fluoresce or only fluoresce slightly and therefore do not affect fluorescence measurements, and the provision of photoinitiators that are bio-based and biocompatible.

A further object of the present invention is to provide surface-structured systems such as decorative surfaces or microfluidic systems based on such embossing varnishes. In addition, surface layers, e.g. decorative surfaces, or microfluidic systems are to be provided which are as bio-based as possible.

### SUMMARY OF THE INVENTION

The problem was solved by providing an embossing varnish containing an aliphatic photoinitiator and an article produced with the embossing varnish with a micro- or nanostructured surface layer.

The object of the present invention is defined in particular in the following points [1] to [15] and [1-1] to [11-2]:

[1] An embossing varnish for micro- or nanostructured surface layers, containing a UV-curable compound with a UV-polymerisable carbon-carbon double bond (abbreviated as C═C double bond in the following) and an aliphatic photoinitiator which contains a moiety selected from an α-diketone or an α-ketocarboxylic acid or salts thereof, particularly metal salts such as sodium salts, or esters thereof. [1-1] Preferably, 90 wt % of the embossing varnish according to point [1] consists of the UV-curable compound and the photoinitiator.

[1-2] The embossing varnish according to point [1] or [1-1] preferably contains 0.1 to 5 parts by weight of the photoinitiator in relation to 100 parts by weight of the UV-curable compound. [1-3] Preferably, an embossing varnish according to one of the preceding points, wherein the photoinitiator comprises a radical selected from a α-ketocarboxylic acid or salts or esters thereof and does not comprise a coinitiator. [1-4] Preferably, in an embossing varnish according to one of the preceding points, the molecular weight of the aliphatic photoinitiator is at most 500 g/mol, preferably at most 300 g/mol.

[2] The embossing varnish according to any one of the preceding points [1] to [1-4], wherein the molecular weight of the UV-curable compound is 200 to 2500 g/mol. [2-1] A combination of the features of points [1], [1-4] and [2] is preferred.

[3] The embossing varnish according to one of the preceding points, wherein the UV-curable compound is an alcohol esterified with one or two or more, preferably two (meth)acrylate groups. [3-1] A combination of the features of points [1], [2-1] and [3] is preferred. [3-2] An embossing varnish according to point [3] is preferred, wherein the alcohol has a molecular weight of 100 to 5000 g/mol, more preferably 100 to 2000 g/mol, and is selected from the group consisting of a hydroxy group-containing biomolecule, a hydroxylated derivative of a biomolecule, a hydroxy group-containing or hydroxylated degradation product of a biomolecule, and an ester or ether of hydroxy group-containing or hydroxylated degradation products of a biomolecule. [3-3] A combination of the features of points [3-1] and [3-2] is preferred.

[4] The embossing varnish according to any of the preceding points, wherein the UV-curable compound is an aliphatic hydroxy group-containing compound esterified with two or more (meth)acrylate groups, wherein the aliphatic hydroxy group-containing compound is selected from the group consisting of a C₆-C₂₄ alcohol, an oligo- or polyether containing C₂-C₆ alkoxy groups, an oligo- or polyester containing a hydroxylated C₂-C₆ mono- or dicarboxylic acid, an oligo- or polyester containing C₂-C₆ alkoxy groups and C₂-C₆ dicarboxylic acid, a hydroxy group-containing polyurethane, in particular a non-isocyanate polyurethane (non-isocyanate PU=NIPU), a glycerol oligomer or polymer, an epoxidised triglyceride of C₆-C₂₄ fatty acids or an epoxidised C₆-C₂₄ fatty acid. Each of these compounds contains at least one hydroxy group and can therefore also be referred to as an alcohol. The at least one hydroxy group can be esterified with a carboxylic acid. [4-1] A combination of the features of points [2-1] and [4] is preferred.

[5] The embossing varnish according to any one of the preceding points, comprising a surface-active non-stick additive which does not fall within the definition of the UV-curable compound and the aliphatic photoinitiator. [5-1] The embossing varnish according to point [5] preferably contains 90 to 99 parts by weight of the UV-curable compound, 0.1 to 5 parts by weight of the photoinitiator and 0.1 to 5 parts by weight of the surface-active non-stick additive, the sum of the parts by weight being 100. [5-2] An embossing varnish according to point [5] or point [5-1] is preferred, wherein the surface-active non-stick additive comprises an optionally branched alkyl radical having at least ten carbon atoms in the main chain or a sugar surfactant consisting of a carbohydrate and a fatty alcohol or a fatty acid.

[6] The embossing varnish according to any one of the preceding points, wherein the UV-curable compound and the photoinitiator are selected such that the extinction coefficient at room temperature and at a wavelength of 365 nm of the photoinitiator in an embossing varnish consisting of the UV-curable compound and 1 wt % of the photoinitiator is at least twice as high as the extinction coefficient of the photoinitiator in a composition consisting of water and 1 wt % of the photoinitiator or in a composition consisting of hexane and 1 wt % of the photoinitiator. [6-1] A combination of the features of points [1], [3] and [6] is preferred. [6-2] A combination of the features of points [1], [3-1] and [6] is more preferred. [6-3] A combination of the features of points [1], [4-1] and [6] is even more preferred.

[7] The embossing varnish according to any one of the preceding points, wherein the UV-curable compound and the photoinitiator are selected such that curing an embossing varnish consisting of the UV-curable compound and 1 wt % of the photoinitiator results in at least 90% of the double bond conversion that occurs when curing an embossing varnish consisting of the UV-curable compound and 1 wt % of α-hydroxy-4-(2-hydroxyethoxy)-α-methylpropiophenone, when a 400 μm thick layer of the respective embossing varnish is cured by exposure under an LED lamp with light of wavelength 365 nm and an intensity of 100 mW for 60 seconds at room temperature. The curing of the embossing varnishes is therefore compared under the same conditions, namely those mentioned. [7-1] A combination of the features of points [1], [3] and [7] is preferred. [7-2] A combination of the features of points [1], [3-1] and [7] is more favoured. [7-3] A combination of the features of points [1], [4-1] and [7] is even more preferred.

[8] An article with a micro- or nanostructured surface layer on a support, wherein the surface layer is an embossed and UV-cured embossing varnish according to one of points [1] to [7-3]. [8-1] The article according to point [8] is preferred which, in particular in the form of a film, is suitable as a microfluidic structure, in particular for cultivating living cells, as a structure with a functional micro- or nanostructured surface, as a structure with an antibacterial, antiviral or antifungal surface or as a structure with an antireflection, flow friction reduction or adhesion effect.

[9] A method of producing an article having a micro- or nanostructured surface layer, preferably as described in point [8] or [8-1], the method comprising applying an embossing varnish according to any one of points [1] to [7-3] to a support and embossing and UV curing the embossing varnish on the support.

[10] The method of point [9], wherein the substrate is a film and the method comprises roll-to-roll embossing.

[11] A method of making an article comprising the following steps (i) to (vi): (i) providing a starting compound having a molecular weight of 100 to 5000 g/mol, preferably 100 to 2000 g/mol, which is a biomolecule or a derivative thereof and is preferably selected from the group consisting of a hydroxy group-containing biomolecule, a hydroxylated derivative of a biomolecule, a hydroxy group-containing or hydroxylated degradation product of a biomolecule, and an ester or ether of hydroxy group-containing or hydroxylated degradation products of a biomolecule; (ii) functionalising the starting compound with a radical-polymerisable group having a C═C double bond, preferably with a (meth)acrylate group, to form a UV-curable compound having a molecular weight of 200 to 5500, preferably 200 to 2500 g/mol; (iv) providing an aliphatic photoinitiator having a moiety selected from a α-diketone or a α-ketocarboxylic acid or salts or esters thereof; (v) preparing a composition comprising the functionalized biomolecule and the aliphatic photoinitiator; and (vi) curing the composition by UV light. [11-1] Preferably, the method according to claim is a method of making an article according to point [8] or [8-1]. [11-2] A method according to point or [11-1] is preferred, wherein the composition obtained in step (v) is an embossing varnish according to any one of points [1] to [7-3].

[12] The method according to any one of points to [11-2], wherein the starting compound is an aliphatic alcohol and the UV-curable compound is an ester of the aliphatic alcohol with one or two or more (meth)acrylate groups.

[13] The method according to any one of points to [12], the method comprising applying the composition obtained in step (v) to a support and, prior to or simultaneously with step (vi), embossing the composition applied to the support, wherein an article having a micro- or nanostructured surface layer is obtained.

[14] An article obtainable by a method according to any one of points [9] to [13].

[15] Use of an article with a micro- or nanostructured surface layer according to point [8] or obtainable by the method according to point as a microfluidic structure, in particular for cultivating living cells, as a structure with a functional micro- or nanostructured surface, as a structure with an antibacterial, antiviral or antifungal surface or as a structure with an antireflection, flow friction reduction or adhesion effect, wherein any of the said structures can be on the surface of a film or the film itself.

### Advantages of the Invention

By using biomaterials in the embossing varnish according to the invention, fossil raw materials can be replaced by bio-based, sustainable and renewable raw materials and the embossing varnish can be produced entirely from renewable raw materials. In particular, the present invention makes it possible for the first time to formulate UV·NIL embossing varnishes based entirely on renewable raw materials. Compared to conventional, aromatic photoinitiators, the photoinitiators according to the invention show little or no fluorescence, since they do not have aromatic rings. The photoinitiators can be advantageously used in microfluidic systems for cultivating living cells, since the photoinitiators occur in the metabolism of the cells and the photoinitiators can even be partially utilised by the cells. Pyruvic acid ethyl ester even has a cytoprotective effect. The photoinitiators can replace conventional petrochemical photoinitiators both in conventional embossing varnishes and in predominantly bio-based embossing varnishes. By using suitable combinations of bio-based UV-curable material and the photoinitiators, the use of coinitiators can be dispensed with. The use of bio-based and biocompatible materials opens up new application possibilities, e.g. in the microfluidics sector as lab-on-foil or lab-on-chip.

### DESCRIPTION OF THE FIGURES

FIG. 1 shows the extinction coefficients of solutions of BTS, EP, KGS and DMKG (1 wt %) in water at a layer thickness of 1 cm.

FIG. 2 shows the absorption spectra of acrylate monomers with a layer thickness of 1 cm in a quartz cuvette, measured against an empty, i.e. air-filled, quartz cuvette.

FIG. 3 shows the extinction coefficients of solutions of pyruvic acid (1 wt %) in water, M2010, M3150, TGDA and M286/H₂O=1/1.

FIG. 4 shows the extinction coefficients of solutions of pyruvic acid ethyl ester (1 wt %) in water, M2010, M3150, TGDA and M286/H₂O=1/1.

FIG. 5 shows the extinction coefficients of solutions of α-ketoglutaric acid (1 wt %) in water, M2010, M3150, TGDA and M286/H₂O=1/1.

FIG. 6 shows the extinction coefficients of solutions of dimethyl-α-ketoglutarate (1 wt %) in water, M2010, M3150, TGDA and M286/H₂O=1/1.

FIG. 7 shows the extinction coefficients of solutions of 4,4-dimethyldihydrofuran-2,3-dione (1 wt %) in water, M2010, M3150, TGDA and M286/H₂O=1/1.

FIG. 8 shows the extinction coefficients of α-ketocarboxylic acids and their esters in the acrylate monomer M2010.

FIG. 9 shows the extinction coefficients of α-ketocarboxylic acids and their esters in the acrylate monomer M3150.

FIG. 10 shows the extinction coefficients of α-ketocarboxylic acids and their esters in the acrylate monomer TGDA.

Fehler! Verweisquelle konnte nicht gefunden werden.Fehler! Verweisquelle konnte nicht gefunden werden.1 shows the photoconversion (DBC: double bond conversion) of the acrylate monomer M2010 determined by ATR-FT-IR spectroscopy as a function of the exposure dose with UV light of wavelength 365 nm.

Fehler! Verweisquelle konnte nicht gefunden werden.Fehler! Verweisquelle konnte nicht gefunden werden.2 shows the photoconversion (DBC: double bond conversion) of the acrylate monomer M3150 determined by ATR-FT-IR spectroscopy as a function of the exposure dose with UV light of wavelength 365 nm.

Fehler! Verweisquelle konnte nicht gefunden werden.Fehler! Verweisquelle konnte nicht gefunden werden.3 shows the photoconversion (double bond conversion or DBC for short) of the acrylate monomer TGDA determined by ATR-FT-IR spectroscopy as a function of the exposure dose with UV light of wavelength 365 nm.

### EMBODIMENTS OF THE INVENTION

The following abbreviations or designations are used in the present invention:
BTS: pyruvic acid
EP: pyruvic acid ethyl ester
KGS: α-ketoglutaric acid
DMKG: α-ketoglutaric acid dimethyl ester
DDFD: 4,4-dimethyldihydrofuran-2,3-dione
EMOB: ethyl-3-methyl-2-oxobutanoate
A2KGS: di-L-arginine-α-ketoglutarate
OKGS: L-ornithine-α-ketoglutarate
M2010: 1,10-decanediol diacrylate
TGDA: triglycerol diacrylate
M3150: triacrylate of ethoxylated trimethylolpropane
M286: polyethylene glycol diacrylate
Sarbio 7101: acrylated epoxidised soybean oil
Rob72: itaconate-containing UV-curing polyester with a bio-renewable carbon content (BRC) of more than 90%
TPO-L: ethyl (2,4,6-trimethylbenzoyl) phenylphosphinate
Irgacure 2959: α-hydroxy-4-(2-hydroxyethoxy)-α-methylpropiophenone
EDMAB: ethyldimethylaminobenzoate

The embossing varnish according to the invention is suitable for micro- or nanostructured surface layers. In other words, it is suitable as a starting material for such layers. The production of such layers involves embossing and hardening the embossing varnish according to the invention as a starting material. For this purpose, the embossing varnish is applied as a surface layer to a support layer. The viscosity of the embossing varnish must therefore be adjusted so that it can be applied easily. Because the embossing varnish is not hardened, i.e. not polymerised, it has the desired viscosity so that it can be applied by brushing or pouring, for example. After application, the embossing lacquer is embossed using an embossing tool. In order to be suitable for the production of micro- or nanostructured surface layers, the embossing varnish according to the invention must therefore have a viscosity within a certain range. Further requirements are that the embossing lacquer according to the invention does not adhere to the embossing tool during embossing and that it can form a stable micro- or nanostructured layer after hardening.

The term “micro- or nanostructured” means that indentations in the micrometer or nanometer range, i.e. from 1 nm to 999 μm, are embossed into the surface of a layer of the embossing varnish. Preferably, the micro- or nanostructured surface layers have indentations from 10 nm to 500 μm. The surface layers have a thickness, i.e. an extent perpendicular to the contact surface with the support, of preferably 10 nm to 1000 μm, more preferably 50 nm to 500 μm, whereby the embossing depth is preferably 50% to less than 100% of the thickness of the surface layer. The term “microfluidics” used herein includes embossing depths in the aforementioned micrometer to nanometer ranges.

The embossing varnish contains at least two components, namely a UV-curable compound and an aliphatic photoinitiator. The two components are structurally different from each other. They are not chemically bonded and are therefore present in the embossing varnish as separate substances. The photoinitiator preferably does not fall under the definition of the UV-curable compound and in this case does not contain a UV-polymerisable C═C double bond, so that the two components differ in this respect. However, the photoinitiator can also fall under the definition of the UV-curable compound and have a polymerisable C═C double bond. In this case, the two components differ from each other in that the UV-curable compound does not fall under the definition of the photoinitiator, i.e. it does not contain a moiety selected from a α-diketone or a α-ketocarboxylic acid or their salts or esters.

In the present invention, the verb “comprise” or a derived form thereof means that a composition preferably comprises said component in an amount of 1 to 100 wt %. It thus includes the meaning that the composition consists of said component. Preferably, the component is the main component and thus contained in an amount of more than 50 wt %. The embossing varnish may contain water. It may be an aqueous dispersion.

In the present invention, singular forms are used to designate components, groups or the like for the sake of simplicity. However, these do not preclude the presence of two or more of the components, groups or the like unless otherwise indicated. For example, the embossing varnish comprises a UV curable compound and an aliphatic photoinitiator. This formulation includes embodiments in which multiple UV-curable compounds and/or multiple aliphatic photoinitiators are included. A UV-curable compound “with a UV-polymerisable C═C double bond” may contain several UV-polymerisable C═C double bonds.

In the context of the invention, the term “oligomer” is used for 2 to 5 consecutive related structural units; a molecular structure with 6 or more such structural units is referred to as a “polymer”.

### UV-Curable Compound

A UV-curable compound used in the embossing varnish according to the invention contains a UV-polymerisable C═C double bond. The compound is UV-curable, i.e. it can be cured with UV light. Curability by other means, e.g. by light of other wavelengths or electron beams, is not excluded. The UV-curable compound is not or not completely polymerised and is converted into the cured coating by radical polymerisation, preferably photopolymerisation using UV light. The UV-curable compound is a monomer or oligomer.

The radical polymerisable groups are non-aromatic C═C double bonds such as vinyl, allyl or norbornenyl groups. Examples are vinyl ethers, allyl ethers, propenyl ethers, alkenes, dienes, unsaturated esters, allyl triazines, allyl isocyanates and N-vinylamides. Preferred UV-curable compounds are (meth)acrylates and derivatives thereof. The term “(meth)acrylate” means “acrylate and/or methacrylate” unless otherwise indicated. The same applies to the expressions “(meth)acrylic acid” and “(meth)acrylate ester”. Examples of (meth)acrylates are 1,4-butanediol dimethacrylate (BDDMA), hexanediol dimethacrylate (HDDMA), 1,3-butylene glycol dimethacrylate (1,3-BGDMA), ethylene glycol dimethacrylate (EGDMA), dodecanediol dimethacrylate (DDDMA), trimethylolpropane trimethacrylate (TMPTMA), trimethacrylate ester (TMA ester), whereby these monomers can be used individually or in combination of two or more.

In addition to the UV-curable compound with a C═C double bond, the embossing varnish can also contain a monomer with two thiol groups, for example glycol di (3-mercaptopropionate) (GDMP). The reaction of the thiol group with a carbon-carbon double bond is a thiol-ene reaction. Further examples of monomer building blocks with at least two thiol groups are 3-mercaptopropionates, 3-mercaptoacetates, thioglycolates and alkylthiols. The embossing varnish according to the invention may contain, for example, a monomer with at least two thiol groups in an amount of from 1 wt % to 50 wt %, in particular from 5 wt % to 30 wt %, the monomer or oligomer having at least one polymerisable double bond in an amount of from 1 wt % to 90 wt %, in particular from 10 wt % to 50 wt %.

The UV-curable compound preferably contains at least one ester of an alcohol and an acid carrying the C═C double bond. More preferably, the UV-curable compound is an alcohol esterified with one or two or more, preferably two (meth)acrylate groups. Alternatively, a (meth)acrylate group can also be coupled via NH₂ or SH to form an amide bond or thioester bond. To link at least two molecules of (meth)acrylic acid, the biomolecule preferably has at least two such groups.

In the present invention, bio-based UV-curable compounds are preferred.

Acrylic acid can be produced from renewable raw materials. For example, a synthesis of acrylic acid from lactic acid is known.

The alcohol esterified with the acid, for example (meth)acrylic acid, is a compound with at least one hydroxyl group and comprises monools and polyols. Polyols are compounds with at least two hydroxyl groups. The alcohol serving as a starting compound for the UV curable compound is selected from the group consisting of a hydroxy group-containing biomolecule, a hydroxylated derivative of a biomolecule, a hydroxy group-containing or hydroxylated degradation product of a biomolecule, and an ester or ether of hydroxy group-containing or hydroxylated degradation products of a biomolecule. Such an alcohol is referred to herein as a bio-based alcohol. It preferably has a molecular weight of 100 to 2000 g/mol. More preferred is α-preferably bio-based-aliphatic alcohol which is a C₆-C₂₄ alcohol, a C₂-C₆ alkoxy group-containing oligo- or polyether, a hydroxylated C₂-C₆ mono- or dicarboxylic acid-containing oligo- or polyester, an oligo- or polyester containing C₂-C₆ alkoxy groups and C₂-C₆ dicarboxylic acid, a NIPU, a glycerol oligomer or polymer, an epoxidised triglyceride of C₆-C₂₄ fatty acids or an epoxidised Co-Cox fatty acid. The alcohols mentioned can serve as a starting compound for the UV-curable compound.

The UV curable compound used in the present invention can be prepared by providing a starting compound and functionalising the starting compound with a group comprising a free radical polymerisable C═C double bond.

The group that has a C═C double bond that can be polymerised by free radicals is preferably a (meth)acrylate group, but can also be an itaconate group, for example. The starting compound can then contain an amine or thio group, so that the coupling takes place via an amide bond or thioester bond. However, a hydroxy group is preferred, so that the starting compound is coupled via an ester group.

Preferred is a starting compound with a molecular weight of 100 to 2000 g/mol, which is preferably selected from the group consisting of
(a) a biomolecule containing hydroxyl groups,
(b) a hydroxylated derivative of a biomolecule,
(c) a hydroxy group-containing or hydroxylated degradation product of a biomolecule; and
(d) an ester or ether of hydroxy-containing or hydroxylated degradation products of a biomolecule.
   (a)

Suitable hydroxy group-containing starting compounds are biomolecules with a molecular weight of 100 to 2000 g/mol, selected from amino acids, peptides, mononucleotides, oligonucleotides, monosaccharides, disaccharides, oligosaccharides or C₆-C₂₄ alcohols, such as mono-, di- or polyols.

(b)

The biomolecules mentioned in (a) or other biomolecules can be provided with one or more hydroxyl groups. For example, triglycerides can be epoxidised or acids can be reduced. C₆-C₂₄ fatty acids can be reduced and, if necessary, epoxidised. The hydroxylated biomolecules have a molecular weight of 100 to 2000 g/mol.

(c)

Biomolecules whose degradation products contain hydroxyl groups or can be hydroxylated are peptides, proteins, oligonucleotides, polynucleotides, disaccharides, oligosaccharides, polysaccharides, triglycerides or fatty acids. Such degradation products can be produced from animal or vegetable oils or fats, i.e. triglycerides from glycerol and fatty acids. The fatty acids are preferably saturated or unsaturated C₈-C₂₄ fatty acids and can be converted into alcohols, preferably polyols, if necessary after epoxidation. After optional hydroxylation, these degradation products have a molecular weight of 100 to 2000 g/mol.

(d)

The starting compound can also be an ester or ether with a molecular weight of 100 to 2000 g/mol from hydroxy group-containing or hydroxylated degradation products of a biomolecule. In the case of an ester from hydroxy group-containing or hydroxylated degradation products of a biomolecule, the degradation products involved in the ester bond additionally contain one or more carboxy groups.

The following degradation products can be produced from biomolecules such as polysaccharides, in particular cellulose or starch:
Diols: ethylene glycol, 1,3-propanediol, 2,3-butanediol, 1,4-butanediol
Polyols: glycerol, pentaerythritol, meso-erythritol, diglycerol
Di- and tricarboxylic acids: citric acid, succinic acid, methyl succinic acid, itaconic acid, fumaric acid, maleic acid, citraconic acid, itaconic anhydride
Hydroxyalkanoic acids: lactic acid, hydroxybutanoic acid
Furans: furan dicarboxylic acid

The degradation products containing hydroxy groups, namely the diols, polyols and hydroxyalkanoic acids, can be used as such or in the form of ethers thereof as starting compounds. The hydroxy group-containing degradation products mentioned can be used as esters with the acids mentioned as starting compounds. Further esters or ethers according to (d) can be prepared from the triglycerides mentioned. The glycerol can be used as polyglycerol. The fatty acids can be esterified with diols, if necessary after additional epoxidation. The esters or ethers according to (d) are in particular oligo- and polyesters and oligo- and polyethers.

Particularly preferred starting compounds with a molecular weight of 100 to 2000 g/mol are C₆-C₂₄ alkyl alcohols, such as mono-, di- or polyols, polyethers such as polyethers containing ethoxy groups, glycerol oligomers such as triglycerol, fatty derivatives such as epoxidised unsaturated triglycerides or optionally epoxidised Ce-Cz fatty acids.

The following are specific examples of UV-curable compounds that can be produced completely bio-based:

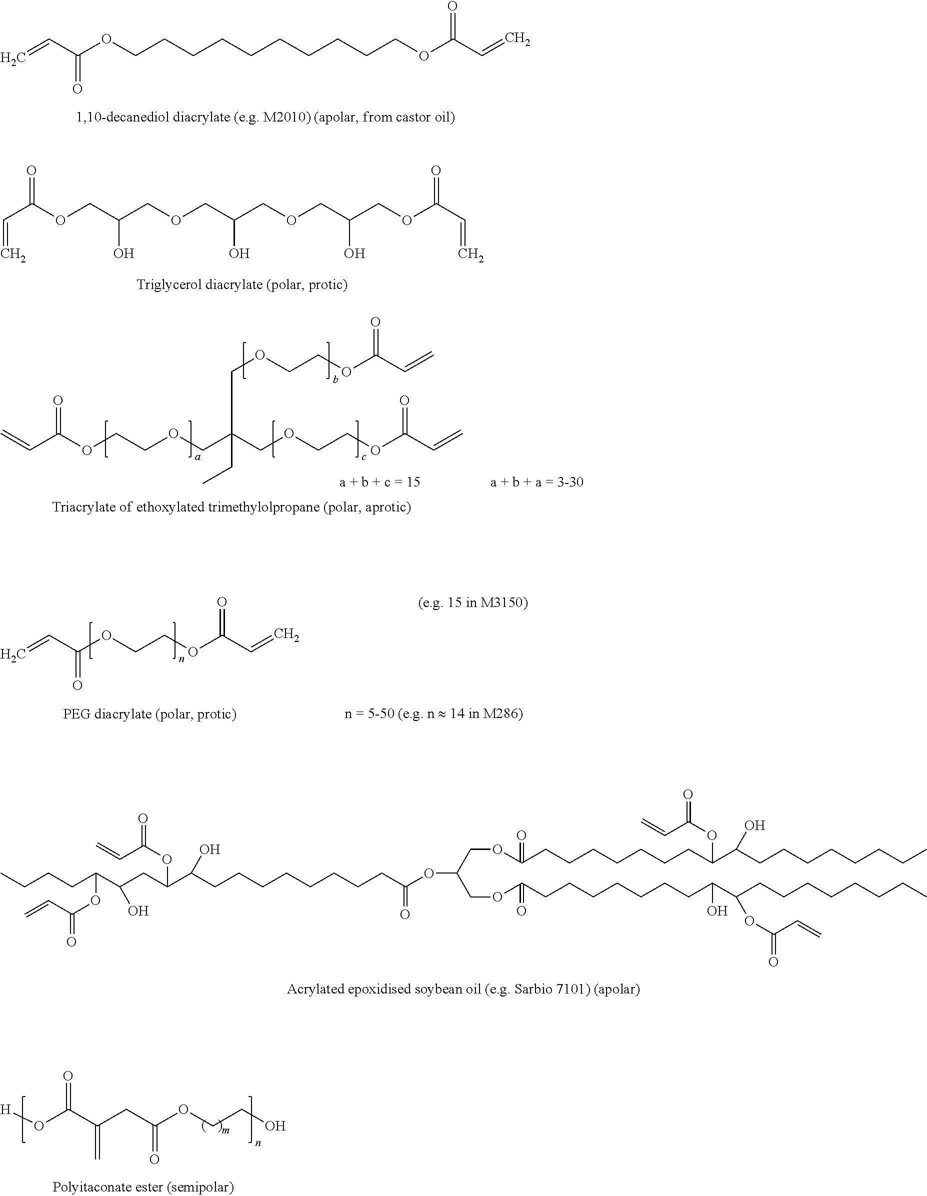

### Photoinitiators

The photoinitiator does not fall under the definition of a UV-curable compound.

The following α-ketocarboxylic acids and their salts, e.g. sodium salts, or esters initiate free radical polymerisation without the addition of co-initiators:

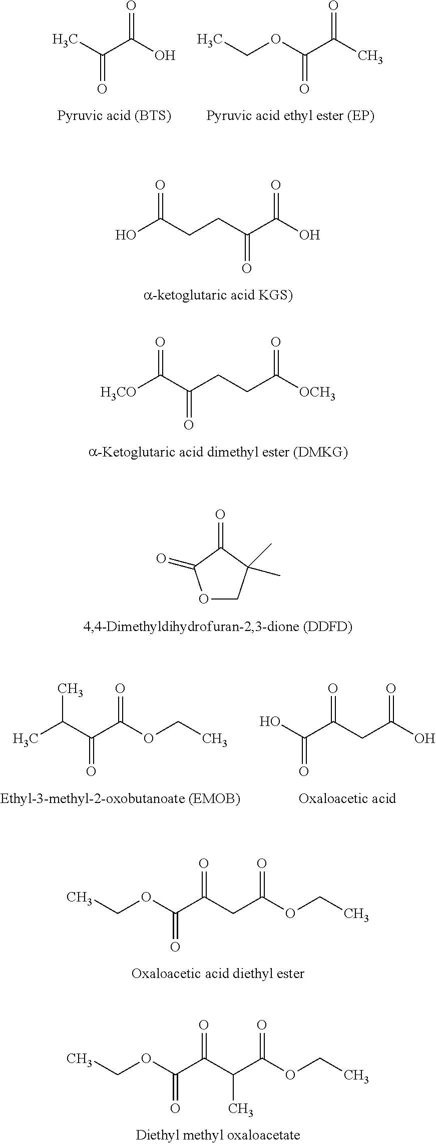

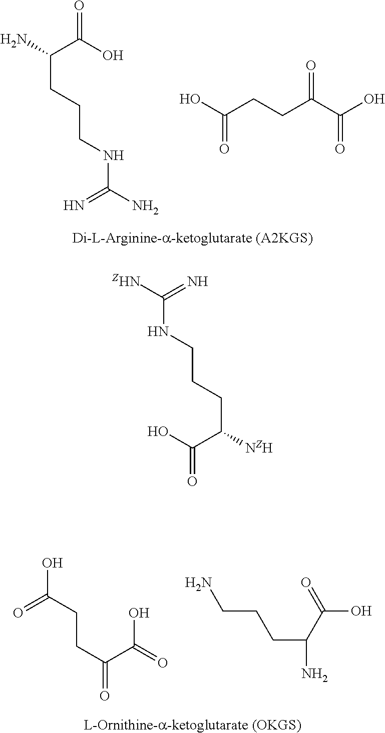

The UV curing of bio-based and conventional embossing varnishes can be started with a series of simple aliphatic α-diones. Examples of such aliphatic α-diones are 2,3-pentanedione, 2,3-hexanedione, 3,4-hexanedione and α-furil. Such α-diketones do not initiate free radical polymerisation very effectively on their own. As Norrish type II photoinitiators, they require co-initiators. The mode of action of Norrish type II photoinitiators is based on the abstraction and intermolecular transfer of a hydrogen atom from a coinitiator, for example a tertiary amine, to the initiator molecule. Thus, in the present invention, a coinitiator is defined by the fact that a hydrogen atom can be abstracted from it and transferred intermolecularly to an initiator molecule. An example of a coinitiator is ethyldimethylaminobenzoate (EDMAB) Furthermore, in the present invention, a coinitiator is defined as not falling within the definition of UV curable compound, photoinitiator and surface active non-stick additive. In embodiments of the invention, the addition of a coinitiator can be omitted since the UV-curable compound or the surface-active non-stick additive can take over its function.

Since the photoinitiators used in the present invention also occur in living cells and are partly intermediate products of cell metabolism or can be utilised by the latter, the photoinitiators not converted during the hardening of the embossing coating do not pose a toxic hazard in applications involving living cells, e.g. in cell cultures. In fact, some of these photoinitiators even serve as nutrients for cells.

### Support

The embossing varnish is applied to a support. There are no particular restrictions with regard to the material of the support. The support can be a polymer substrate, for example a film.

The support can be bio-based. Films based on cellulose and polylactate are commercially available.

### Surface-Active Non-Stick Additive

In order to reduce or completely prevent adhesion of the embossing varnish to the embossing tool, the embossing varnish according to the invention may contain a surface-active non-stick additive. The surface-active non-stick additive does not fall under the definition of the UV-curable compound and the aliphatic photoinitiator. It can be silicone-containing or fluorine-containing. In particular, the additive is at least one member selected from the group comprising silicone-containing or fluorine-containing additives. Specific examples are non-ionic surfactants such as polyether siloxanes, fatty alcohol ethoxylates such as polyoxyethylene (9)-lauryl ether, monofunctional polydimethylsiloxane polyethoxy (meth)acrylates, alkyl (meth)acrylates, perfluoroalkyl (meth)acrylates and perfluoropolyether (meth)acrylates. Amphiphilic alkyl-containing, silicone-containing or fluorine-containing additives contribute to reducing adhesion and facilitating the release of the embossing lacquer from the embossing tool, whereby the perfluorinated additives have proven to be particularly favourable and reliably enable a plurality of impressions of a pattern. The at least one additive can be contained in the starting varnish in an amount of 0.1 to 3 wt %. Adhesion of the embossing varnish to the embossing tool can also be prevented by modifying the surface of the embossing tool, in particular the embossing die, with regard to its hydrophobicity.

### Method

The embossing varnish is applied to a support layer, e.g. a polymer substrate. Preferably, embossing varnish has a desired viscosity so that it can be applied by brushing or moulding, for example. In the roll-to-roll process, the embossing varnish is applied to the substrate, for example using a slot die or by gravure printing with an engraving roller. A micro- or nanostructured stamp with the inverted profile of the desired micro- or nanostructured surface is used as a negative mould and pressed into the embossing lacquer, in which the desired structure is then embossed as a positive mould. The embossing die can be made of metal, for example nickel, or of polymer materials, whereby polymer materials have potentially lower surface energies than nickel, which reduces paint adhesion during the embossing process. When exposed to UV light, the embossing varnish polymerises and becomes solid. After separating the stamp from the embossed pattern, the profile of the stamp is replicated in inverted form. When using a continuous roll-to-roll process, the cylindrical stamp is part of a roll. This allows very large areas to be structured in a short time.

### EXAMPLES

Various bio-based UV-curable compounds and photoinitiators were investigated. The present invention is explained in more detail below on the basis of the test results shown in the figures.

FIG. 1 shows that the extinction coefficients of various α-ketocarboxylic acids and their esters in water are similar. The α-ketocarboxylic acids and their esters absorb in the UV-A spectral range of interest for UV curing (λ=380-315 nm). The extinction coefficients are not high and are usually in the single-digit range between λ=380-315 nm. This absorption in UV-A is based on an n-π* transition. In the UV-C range at λ<280 nm, these molecules absorb more strongly due to a π-π* transition. However, this spectral range is of little interest for UV curing because (meth)acrylates already absorb there themselves.

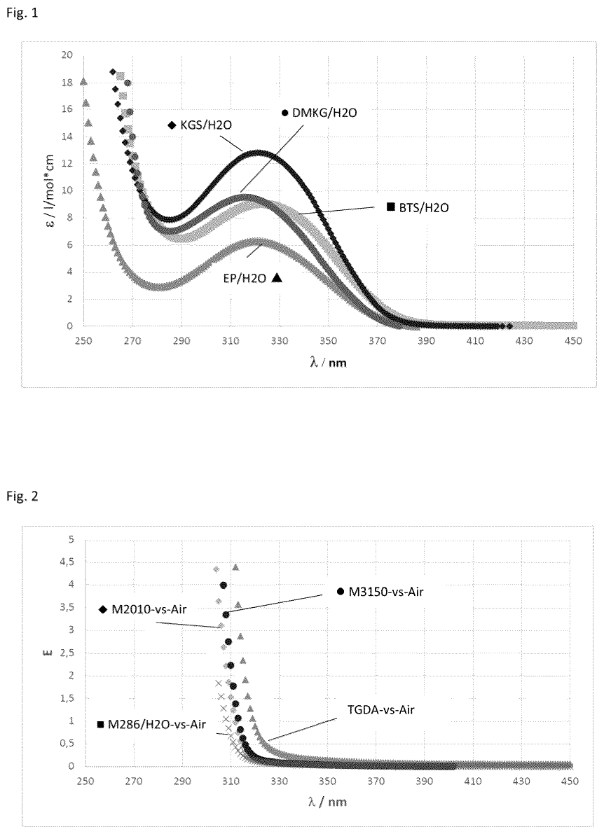

FIG. 2 shows that M2010, M3150 and M286 show a very steep absorption edge at around λ=310 nm and TGDA at %=320 nm. Absorption is very strong in the wavelength range below 310 nm and 320 nm. Because the absorption of the photoinitiators at shorter wavelengths would be completely overshadowed by the strong absorption of the monomers, the following UV absorption spectra and extinction coefficients of the photoinitiators in these acrylate monomers are only shown in the wavelength range from 450 to 300 nm.

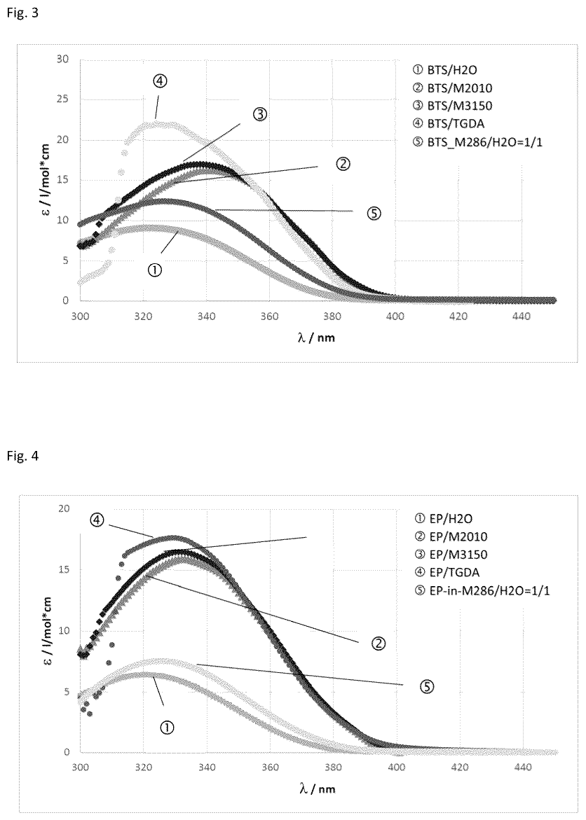

FIG. 3 shows that the absorption of pyruvic acid (BTS) in the UV-A range is significantly stronger in all three acrylate monomers than in water. The absorption maximum of the n-π* transition is clearly bathochromically shifted (red shift) both in the apolar M2010 with its aliphatic decane backbone and in the polar M3150 with its polyether backbone. While the maximum absorption of this excitation in water occurs at 322 nm, the maximum in M2010 and M3150 is at 340 nm. This is very favourable for the UV curing of embossing varnishes. In aqueous polyethylene glycol diacrylate M286/H₂O=1/1 the absorption is stronger than in water and slightly bathochromic shifted and is therefore almost averaged between water and the M286 chemically similar M3150. BTS absorbs most strongly in triglycerol diacrylate. In this polar and protic medium, the maximum absorption is similar to that in water at around 320 nm. Apparently, the ability of the medium to donate protons plays a greater role in the n-π* transition of BTS than the pure polarity.

FIG. 4 shows that pyruvic acid ethyl ester (EP) absorbs much more strongly in all three acrylate monomers analysed than in water. The extinction coefficients of EP in M2010 and M3150 are almost identical to those of BTS. The polarity of the medium does not play a significant role here either. The absorption maximum of the n-π* band of EP is hardly shifted in TGDA. Therefore, the ability of TGDA to protonate does not appear to have a significant influence on the n-π* absorption of EP. In aqueous polyethylene glycol diacrylate M286/H₂O=1/1 the absorption is hardly stronger than in water and slightly bathochromically shifted.

The investigations show that for the favourable bathochromic shift of the n-π* transition, the ability of the medium to proton donate plays a greater role than pure polarity.

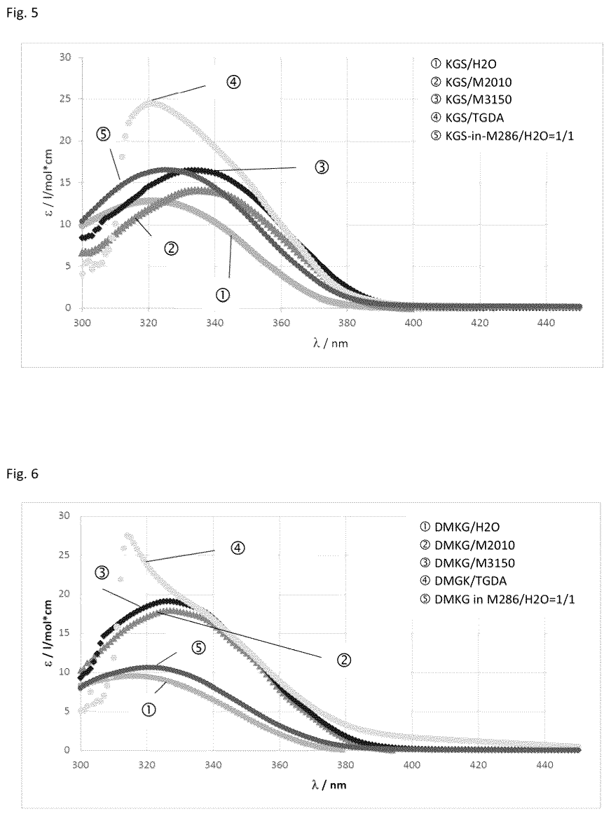

FIG. 5 shows that in solutions of α-ketoglutaric acid (KGS), the intensity of the n-π* band in M2010 and M3150 is also slightly higher than in water and again shifted bathochromically by around 20 nm. In the protic medium TGDA, the intensity of the absorption is significantly higher and the absorption maximum is at almost the same wavelength as in water. With the exception of the slightly stronger absorption in water, the extinction coefficients of KGS are very similar to those of pyruvic acid in all the media analysed. In aqueous polyethylene glycol diacrylate M286/H₂O=1/1, the absorption strength of KGS is similar to that of the chemically similar M286 M3150. The position of the absorption maximum is almost exactly in the centre between water and M3150, which also has a PEG backbone.

FIG. 6 shows that the spectra of dimethyl-α-ketoglutaric acid (DMKG) in the non-polar M2010 and the polar M3150 are identical. In both cases, the absorption of the n-π* transition is more than twice as strong as in water and shifted bathochromically by about 20 nm in each case. The spectra of these two esters DMKG and EP are therefore very similar. The absorption strength in the M286/H₂O=1/1 system is similar to that in water. The absorption maximum is bathochromically shifted by approx. 6 nm.

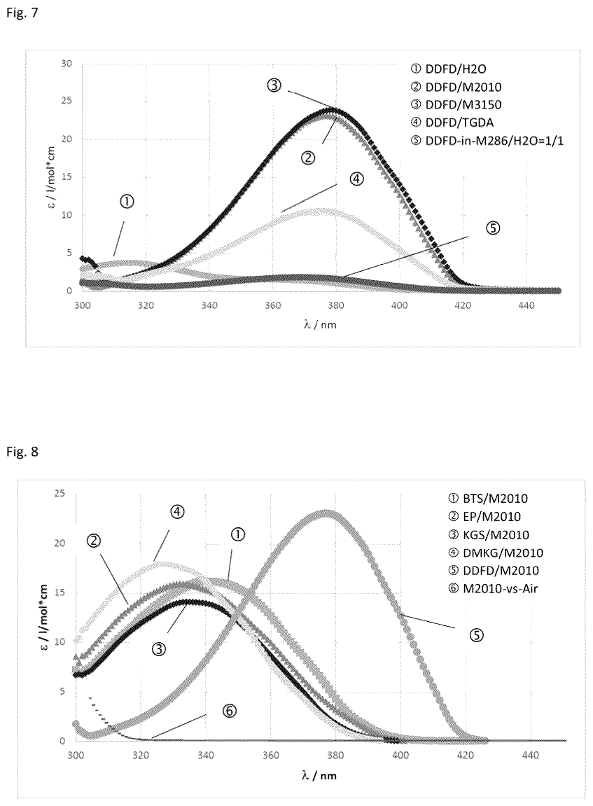

FIG. 7 shows that the absorption maximum of the n-π* transition of dimethyldihydrofuran-2,3-dione (DDFD) is clearly bathochromically shifted to around 375 nm in all acrylate monomers. This is due to the coplanar fixation of the carbonyl oxygen atoms in the cyclic DDFD and the resulting increased conjugation of the n-orbitals. The intensity of the n-π* transition is significantly higher in the two aprotic monomers M2010 and M3150 than in the protic TGDA. The extinction coefficients of DDFD in water and M286/H₂O=1/1 are out of the ordinary. It is possible that DDFD saponifies in water. This would explain the low long-wavelength band at 380 nm and the absorption at 320 nm.

Table 1 summarises the results illustrated in FIGS. 1 to 7. Table 1 shows the wavelength of the absorption maximum, the maximum extinction coefficient and the extinction coefficient at 365 nm of solutions of the investigated a ketocarboxylic acids and their esters in the acrylate monomers M2010, M3150 and TGDA as well as in water and n-hexane as extreme references, namely polar/protic and apolar/aprotic. In addition, solutions of the described α-ketocarboxylic acids and their esters as well as the two amino acid α-ketoglutaric acid complexes di-L-arginine α-ketoglutarate (A2KGS) and L-ornitine α-ketoglutarate (OKGS) were also analysed in mixtures of polyethylene glycol diacrylate (PEGDA) (Miramer M268, M=600 g/mol) and water (with a weight ratio M286/H₂O=1/1). Stable hydrogels are formed from the aqueous PEGDA solutions of the aforementioned α-ketocarboxylic acids and their derivatives by UV irradiation. The investigated α-ketocarboxylic acids and their esters as well as the two highly water-soluble amino acid complexes also initiate radical polymerisation very effectively in an aqueous medium.

TABLE 1

| | λₘₐₓ | ∈ₘₐₓ | ∈ at 365 nm |
|---|---|---|---|
| | [nm] | [l/mol*cm] | [l/mol*cm] |
| BTS | | | |
| H₂O | 322 | 9.0 | 2.5 |
| Hexane | 352 | 3.8 | 2.6 |
| M2010 | 341 | 16.2 | 10.5 |
| M3150 | 338 | 16.9 | 10.3 |
| TGDA | 321 | 21.8 | 9.4 |
| M286/H₂O = 1/1 | 327 | 12.3 | 4.8 |
| EP | | | |
| H₂O | 321 | 6.3 | 1.3 |
| Hexane | 334 | 16.0 | 10 |
| M2010 | 332 | 15.9 | 8 |
| M3150 | 331 | 16.5 | 8.1 |
| TGDA | 330 | 17.6 | 7.5 |
| M286/H₂O = 1/1 | 325 | 7.5 | 2.3 |
| KGS | | | |
| H₂O | 321 | 12.8 | 2.6 |
| Hexane | 350 | 0.06* | hardly |
| | | | dissolved |
| M2010 | 335 | 14.1 | 6.8 |
| M3150 | 333 | 16.5 | 7.8 |
| TGDA | 321 | 24.4 | 7.6 |
| M286/H₂O = 1/1 | 325 | 16.5 | 5 |
| A2KGS M286/H₂O = 1/1 | 307 | 31.2 | 7 |
| OKGS M286/H₂O = 1/1 | 298 | 17.3* | 5.1 |
| DMKG | | | |
| H₂O | 316 | 9.5 | 1.2 |
| Hexane | 328 | 9.0 | 3.7 |
| M2010 | 327 | 17.9 | 5.9 |
| M3150 | 327 | 19.1 | 6.3 |
| TGDA | 315 | 27.3* | 7.1 |
| M286/H₂O = 1/1 | 322 | 10.6 | 2.2 |
| DDFD | | | |
| H₂O | 370 | 1.1 | 1.2 |
| Hexane | 381 | 4.16 | incompletely |
| | | | dissolved |
| M2010 | 377 | 23.0 | 20.4 |
| M3150 | 372 | 22.9 | 20.6 |
| TGDA | 375 | 10.6 | 9.8 |
| M286/H₂O = 1/1 | 369 | 1.8 | 1.8 |

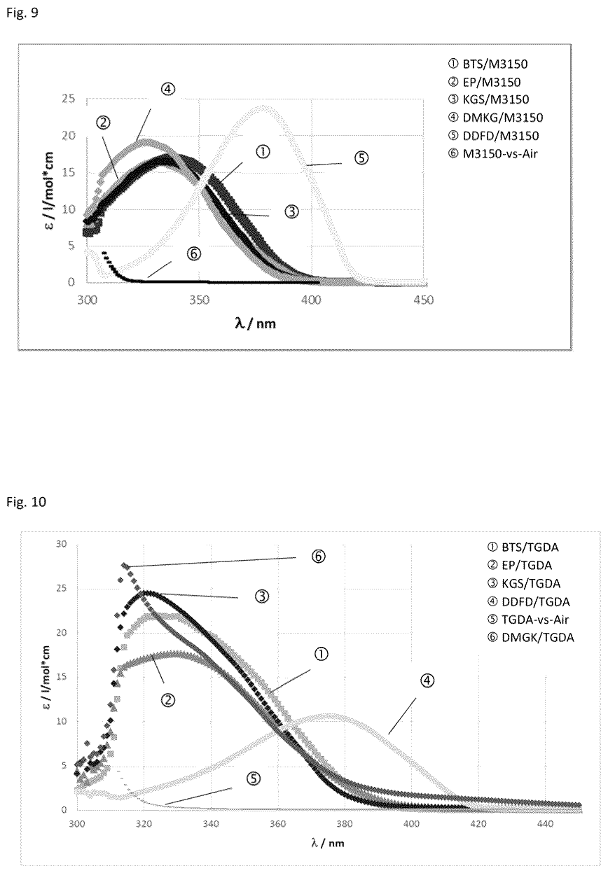

FIGS. 8 and 9 show that the spectra of the analysed α-ketocarboxylic acids and their esters, with the exception of DDFD in the acrylate monomers M 2010 and M3150, are very similar both in absorption position and strength. The strong bathochromic shift of DDFD is due to the coplanar fixation of the carbonyl oxygen atoms as described above. DMKG shows a small hypsochromic shift (blue shift) of about 10 nm compared to BTS, EP and KGS.

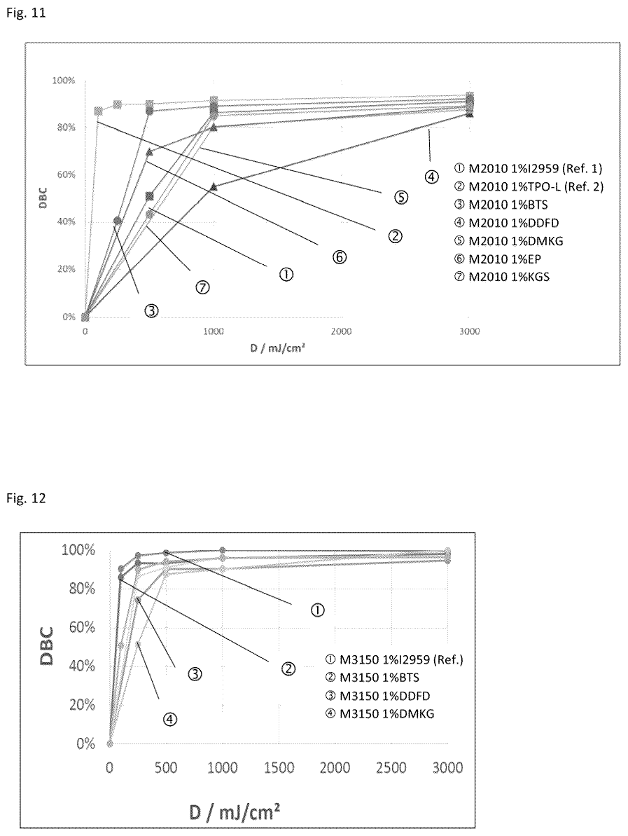

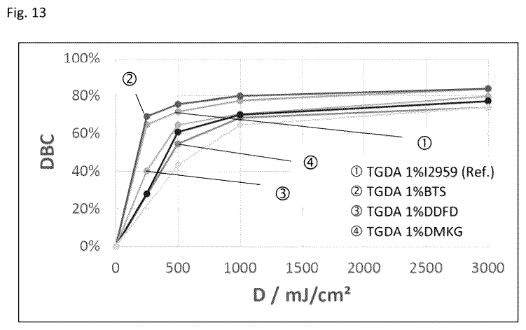

FIG. 10 shows that the absorption strength in TGDA is significantly lower compared to BTS. EP and KGS. The hypsochromic shift of DMKG also observed here means that the absorption maximum already falls within the strong absorption of the medium TGDA and can therefore no longer be resolved.

### ATR-FT-IR Studies on the Efficiency of Photoinitiators

Embossing varnishes containing acrylate monomers and 1 wt % photoinitiator were exposed between two glass plates in layer thicknesses of 400 μm (with 400 μm thick spacers) under an LED lamp with UV-A light of wavelength 365 nm at an intensity of 100 mW.

The results are shown in FIGS. 11 to 13.

FIG. 11 shows the results of curing M2010 with various photoinitiators. The conventional aromatic photoinitiators TPO-L and Irgacure 2959 were used as references. TPO-L, which is known to be highly efficient, showed the highest efficiency, followed by pyruvic acid (BTS) and Irgacure 2959. Pyruvic acid ethyl ester (EP) also starts the polymerisation faster than Irgacure 2959, but the final double bond conversion is slightly lower than with Irgacure 2959. KGS and its ester DMKG show similar efficiency to Irgacure 2959, and only the cyclic ester DDFD initiates the photopolymerisation of M2010 slightly slower. The maximum double bond conversion is 90% for all photoinitiators.

FIG. 12 shows that the photopolymerisation reaction with all photoinitiators tested is significantly faster in M3150 than in M2010 and that the acrylate double bonds are almost completely converted. Irgacure 2959 starts the fastest here, but pyruvic acid and α-ketoglutaric acid are almost as fast. The corresponding esters EP and DMKG as well as the cyclic ester DDFD initiate the photopolymerisation of M3150 somewhat more slowly.

FIG. 13 shows that pyruvic acid is the most effective initiator of photopolymerisation in TGDA, even before the reference Irgacure 2959. The cyclic ester DDFD is more effective than the esters EP and DMKG. KGS is the least effective in TGDA. The maximum achievable double bond conversion in TGDA with BTS and Irgacure 2959 is 84% and 83% respectively. With the other initiators, 75 to 80% are achieved.

The differences in the maximum double bond conversion could be due to the different glass transition temperatures of the acrylate monomers. M3150 has a very low glass transition temperature of −31° C. No solidification occurs during photopolymerisation at room temperature, so that the reactive radicals and monomers are not frozen and thus a high polymerisation conversion is achieved. M2010 has a glass transition temperature of 36° C., so that the mobility of the radicals is restricted during polymerisation, which leads to a slightly lower final double bond conversion. The high number of hydrogen bonds in triglycerol diacrylate probably increases the glass transition temperature, which would explain the lower maximum double bond conversion.

Tests were carried out on the UV curing of different monomers using different photoinitiators. In each case, solutions containing 1 wt % of the photoinitiators were exposed between two glass plates in layer thicknesses of 400 μm (with 400 μm thick spacers) under an LED lamp with UV-A light of wavelength 365 nm or 395 nm with an intensity of 100 mW for different lengths of time. The consistency of the UV-polymerised films was then evaluated qualitatively. The results are summarised in Table 2 and Table 3.

TABLE 2

| | Photo- | Exposure | |
|---|---|---|---|
| Monomer | initiator | time | Result |
| | BTS | | |
| M2010 | 1% | 60 s | elastic film |
| M3150 | 1% | 60 s | elastic film |
| TGDA | 1% | 60 s | hard film |
| Rob 72 | 1% | 60 s | elastic film |
| Sarbio7101 | 1% | 60 s | elastic film |
| IBOA (isobornyl | 1% | 60 s | glass-hard adhesive |
| acrylate) | | | |
| M286/H₂O = 1/1 | 0.5% | 30 s | hydrogel film |
| M286/H₂O = 1/1 | 0.5% | 60 s | stable hydrogel film |
| | EP | | |
| M2010 | 3% | 30 s | elastic film |
| M3150 | 3% | 30 s | elastic film |
| TGDA | 3% | 30 s | hard film |
| Sarbio 7101 | 3% | 30 s | elastic film |
| Rob72 | 3% | 30 s | elastic film |
| Ebecryl 4820 | 2% | 30 s | solid film |
| M286/H₂O = 1/1 | 0.5% | 30 s | hydrogel film |
| M286/H₂O = 1/1 | 0.5% | 60 s | stable hydrogel film |
| | KGS | | |
| M2010 | 3% | 30 s | elastic film |
| M3150 | 3% | 30 s | elastic film |
| TGDA | 3% | 30 s | hard film |
| | O₂-KGS | | |
| M286/H₂O = 1/1 | 1% | 30 s | hydrogel film |
| M286/H₂O = 1/1 | 1% | 60 s | stable hydrogel film |
| | A₂-KGS | | |
| M286/H₂O = 1/1 | 1% | 30 s | hydrogel film |
| M286/H₂O = 1/1 | 1% | 60 s | stable hydrogel film |
| | DMKG | | |
| M2010 | 3% | 60 s | elastic film |
| M2010 | 3% | 15 s | elastic film |
| TGDA | 2% | 60 s | hard film |
| TGDA | 2% | 15 s | hard film |
| M3150 | 2% | 60 s | elastic film |
| M3150 | 2% | 15 s | elastic film |
| IBOA (isobornyl | 1% | 60 s | brittle hard film |
| acrylate) | | | |
| Rob72 | 3% | 180 s | solid film |
| Rob72 | 3% | 60 s | elastic film |
| | EMOB | | |
| M2010 | 2% | 30 s | elastic film |
| M2010 | 2% | 15 s | elastic film |
| M2010 | 2% | 5 s | crumbly film |
| M2010 | 1% | 30 s | elastic film |
| M2010 | 1% | 15 s | elastic film |
| M2010 | 1% | 5 s | very crumbly film |
| Sarbio7101 | 1% | 30 s | elastic film |
| Sarbio7101 | 1% | 15 s | elastic film |
| Sarbio7101 | 1% | 5 s | crumbly film |
| Rob72 | 1% | 15 s | liquid |
| Rob72 | 1% | 60 s | crumbly film |
| | DDFD | | |
| Rob72 | 2% | 60 s | elastic film |
| Rob72 | 2% | 30 s | crumbly film |
| Sarbio 7101 | 2% | 30 s | elastic film |
| Sarbio 7101 | 2% | 15 s | elastic film |
| Sarbio 7101 | 2% | 5 s | crumbly film |
| | DEOA | | |
| M2010 | 1% | 30 s | liquid |
| M2010 | 1% | 60 s | less stable film |
| M3150 | 1% | 30 s | less stable film |
| M3150 | 1% | 60 s | elastic film |
| TGDA | 1% | 30 s | elastic film |
| TGDA | 1% | 60 s | solid film |
| | DEMOA | | |
| M2010 | 1% | 30 s | less stable film |
| M2010 | 1% | 60 s | moderately stable |
| | | | film |
| M3150 | 1% | 30 s | very elastic film |
| M3150 | 1% | 60 s | elastic film |
| TGDA | 1% | 30 s | elastic film |
| TGDA | 1% | 60 s | solid film |
| | OES | | |
| M2010 | 1% | 30 s | unstable film |
| M2010 | 1% | 60 s | very elastic film |
| M3150 | 1% | 30 s | elastic film |
| M3150 | 1% | 60 s | elastic film |
| TGDA | 1% | 30 s | solid film |
| TGDA | 1% | 60 s | hard film |

TABLE 3

| | Photo- | Wave- | Exposure | |
|---|---|---|---|---|
| Monomer | initiator | length | time | Result |
| | 2,3-PDO | | | |
| TGDA | 1% | 365 nm | 60 s | hard film |
| TGDA | 1% | 365 nm | 30 s | hard film |
| TGDA | 1% | 365 nm | 15 s | elastic film |
| TGDA | 1% | 395 nm | 60 s | hard film |
| TGDA | 1% | 395 nm | 30 s | elastic film |
| M3150 | 1% | 365 nm | 60 s | crumbly film |
| M2010 | 1% | 365 nm | 30 s | crumbly film |
| M2010 | 1% | 365 nm | 60 s | crumbly film |
| Sarbio7101 | 2% | 365 nm | 60 s | crumbly film |
| Rob72 | 2% | 365 nm | 60 s | merely gels |
| | 3,4-HDO | | | |
| TGDA | 3% | 395 nm | 60 s | solid film |
| TGDA | 3% | 365 nm | 60 s | solid film |
| TGDA | 1.5% + 0.5% | 395 nm | 60 s | solid film |
| | EDMAB | | | |
| TGDA | 1.5% + 0.5% | 365 nm | 60 s | solid film |
| | EDMAB | | | |
| M2010 | 3% | 365 nm | 30 s | liquid |
| M2010 | 3% | 395 nm | 30 s | gelled |

What is claimed is:

1. An article with a micro- or nanostructured surface layer on a support, wherein the surface layer is obtainable by embossing and UV-curing an embossing varnish, wherein the embossing varnish contains a UV-curable compound with a UV-polymerisable C═C double bond and an aliphatic photoinitiator containing a moiety selected from an α-diketone or an α-ketocarboxylic acid or salts or esters thereof.

2. The article according to claim 1, wherein the molecular weight of the UV curable compound in the embossing varnish is 200 to 2500 g/mol.

3. The article according to claim 1, wherein the UV-curable compound in the embossing varnish is an alcohol esterified with one or two or more (meth)acrylate groups.

4. The article according to claim 1, wherein the UV curable compound in the embossing varnish is an aliphatic hydroxy group-containing compound esterified with two or more (meth)acrylate groups, wherein the aliphatic hydroxy group-containing compound is a C6-C24 alcohol, an oligo- or polyether containing C2-C6 alkoxy groups, an oligo- or polyester containing a hydroxylated C2-C6 mono- or dicarboxylic acid, an oligo- or polyester containing C2-C6 alkoxy groups and C2-C6 dicarboxylic acid, a non-isocyanate-based polyurethane, a glycerol oligomer or polymer, an epoxidised triglyceride of C6-C24 fatty acids or an epoxidised C6-C24 fatty acid.

5. The article according to claim 1, wherein the embossing varnish contains a surface active non-stick additive.

6-8. (canceled)

9. A method for producing an article having a micro- or nanostructured surface layer, which comprises applying an embossing varnish as recited in claim 1 to a substrate and embossing and UV-curing the embossing varnish on the substrate.

10. The method according to claim 9, wherein the support is a film and the method comprises roll-to-roll embossing.

11. A method of manufacturing an article comprising the following steps:
(i) providing a starting compound having a molecular weight of 100 to 2000 g/mol, which is a biomolecule or a derivative thereof and is preferably selected from the group consisting of a hydroxy group-containing biomolecule, a hydroxylated derivative of a biomolecule, a hydroxy group-containing or hydroxylated degradation product of a biomolecule and an ester or ether of hydroxy group containing or hydroxylated degradation products of a biomolecule,
(ii) functionalising the starting compound with a radical-polymerisable group having a C═C double bond, preferably with a (meth)acrylate group, to form a UV-curable compound with a molecular weight of 200 to 2500 g/mol,
(iii) providing an aliphatic photoinitiator having a moiety selected from a α-diketone or a α-ketocarboxylic acid or salts or esters thereof,
(iv) preparing a composition comprising the functionalized biomolecule and the aliphatic photoinitiator; and
(v) curing the composition by means of UV light,
wherein the method comprises applying the composition obtained in step (iv) to a support and, prior to or simultaneously with step (v), embossing the composition applied to the support, whereby an article having a micro- or nanostructured surface layer is obtained.

12. The method according to claim 11, wherein the starting compound is an aliphatic alcohol and the UV-curable compound is an ester of the aliphatic alcohol with one or two or more (meth)acrylate groups.

13. (canceled)

14. An article obtainable by a method according to claim 9.

15. The use of an article with a micro- or nanostructured surface layer according to claim 1 as a microfluidic structure, in particular for the cultivation of living cells, as a structure with a functional micro- or nanostructured surface, as a structure with an antibacterial, antiviral or antifungal surface, or as a structure with an antireflection, flow friction reduction or adhesion effect.
